(19) United States
(12) Patent Application Publication
(10) Pub. No.: US 2014/0343141 A1
(43) Pub. Date: Nov. 20, 2014

(54) TOPICAL COMPOSITION COMPRISING AN INGENOL DERIVATIVE AND A SURFACTANT-COSOLVENT MIXTURE

(71) Applicant: LEO LABORATORIES LIMITED, Dublin (IE)

(72) Inventors: Per Ola Arvidsson, Ballerup (DK); Edit Farkas, Ballerup (DK); Karsten Petersson, Ballerup (DK)

(21) Appl. No.: 14/364,540

(22) PCT Filed: Dec. 12, 2012
(86) PCT No.: PCT/IB2012/057254

§ 371 Date: Jun. 11, 2014

Related U.S. Application Data
(60) Provisional application No. 61/569,635, filed on Dec. 12, 2011.

(51) Int. Cl. A61K 47/10; A61K 9/00; A61K 9/107; A61K 31/22; A61K 47/44

(52) U.S. Cl. CPC A61K 47/10; A61K 31/22; A61K 47/44; A61K 9/107; A61K 9/0014
USPC 514/511

(57) ABSTRACT

A topical composition for cutaneous application which is a water-in-oil emulsion comprises an oily phase comprising (a) an ingenol derivative in dissolved form; (b) at least one non-ionic surfactant selected from the group consisting of polyoxyl glycerides, polyoxyethylene castor oil derivatives, polyoxyethylene alkyl ethers, polysorbates, or a mixture of acrylamide acryloyldimethyl taurate copolymer, isohexadecane and polysorbate 80, sterols, fatty alcohols, fatty acid phosphonates, mono- or diglycol esters, mono- di- or polyglyceryl esters, mono-, di- or plyglucose esters, sucrose esters or sorbitan esters, the non-ionic surfactant being present in an amount of from about 0.5% by weight to about 10% by weight of the composition; (c) a solvent for the ingenol derivative; and an aqueous phase buffered to a pH of 2.6-3.7.

### FIELD OF INVENTION

The present invention relates to a topical pharmaceutical formulation comprising a pharmacologically active agent, a surfactant, a cosolvent and an aqueous phase.

### BACKGROUND OF THE INVENTION

The invention provides a pharmaceutical formulation suitable for topical application of the compound ingenol-3-angelate (2-methyl-2(Z)-butenoic acid (1aR,2S,5R,5aS,6S,8aS,9R,10aR)-5,5a-dihydroxy-4-(hydroxymethyl)-1,1,7,9-tetramethyl-11-oxo-1a,2,5,5a,6,9,10,10a-octahydro-1H-2,8a-methanocyclopenta[a]cyclopropa[e]cyclodecen-6-yl ester; PEP005). Ingenol-3-angelate (PEP005) is a protein kinase C activator in phase III clinical development for the treatment of actinic keratosis. The drug candidate is also in phase II trials for non-melanoma skin cancer [Ogbourne, S. M.; Anti-cancer Drugs, (2007), 18, 357-62].

The compound ingenol-3-angelate (PEP005) [Sayed, M. D. et. al.; Experienta, (1980), 36, 1206-1207] can be isolated from various Euphorbia species, and particularly from Euphorbia peplus [Hohmann, J. et. al; Planta Med., (2000), 66, 291-294] and Euphorbia drummondii by extraction followed by chromatography as described in U.S. Pat. No. 7,449,492. Pharmaceutical formulation of the compound has been described in WO200768963.

Angelic acid and angelic acid esters such as ingenol-3-angelate, are prone to isomerisation of the double bond to form the tiglate ester, particularly at basic pH or when subjected to heat [Beeby, P., Tetrahedron Lett. (1977), 38, 3379-3382, Hoskins, W. M., J. Chem. Soc. Perkin Trans. 1, (1977), 538-544, Bohlmann, F. et. al., Chem. Ber. (1970), 103, 561-563]. As a consequence only carefully optimised conditions for ester formation can be applied in the synthetic preparation of ingenol-3-angelate.

Furthermore, ingenol-3-acylates are known to be unstable as they rearrange to afford the ingenol-5-acylates and ingenol-20-acylates [Sorg, B. et. al, Z. Naturforsch., (1982), 37B, 748-756].

WO 2007/068963 discloses a gel formulation for the treatment of skin cancer in which ingenol angelate is dissolved in an aprotic solvent, the formulation further comprising an acidifying agent such that the pH of the formulation is no greater than 4.5. The aqueous gel is generally stored at refrigeration temperature.

One object of the invention is therefore to provide a composition of the ingenol derivative which is stable at room temperature for the entire shelf-life of the composition.

Another object of the invention is to provide a composition exhibiting favourable penetration characteristics and biological activity.

A further object of the invention is to provide a composition with reduced skin irritation and favourable cosmetic properties and improved patient compliance.

### SUMMARY OF THE INVENTION

In the research leading to the present invention, it was an object to identify a solvent mixture which is as effective at dissolving compounds such as ingenol derivatives as low-molecular alcohols or diols when used on their own as co-solvents in admixture with an aqueous phase. It has surprisingly been found that mixing certain surfactants with certain oily solvents provides mixtures with an exceptionally high solubilization capacity. The resulting composition where the individual solvent components act synergistically leads to a satifactory penetration of the ingenol derivative into the viable layers of the skin.

In one aspect, the present invention relates to a topical composition for cutaneous application which is a water-in-oil emulsion comprising an oily phase comprising

(a) an ingenol derivative in dissolved form;
(b) at least one non-ionic surfactant selected from the group consisting of polyoxyl glycerides, polyoxyethylene castor oil derivatives, polyoxyethylene alkyl ethers, polysorbates, or a mixture of acrylamide acryloyldimethyl taurate copolymer, isohexadecane, polysorbate 80, sterols, fatty alcohols, fatty acid phosphonates, mono- or diglycol esters, mono- di- or polyglyceryl esters, mono-, di- or polyglucose esters, sucrose esters or sorbitan esters, the non-ionic surfactant being present in an amount of from about 0.5% by weight to about 10% by weight of the composition;
(c) a solvent for the ingenol derivative; and
an aqueous phase buffered to a pH of 2.6-3.7.

Ingenol derivatives such as ingenol-3-angelate are known to be extremely sensitive to higher pH conditions (pH above about 4.5 in aqueous compositions or alkaline reacting substances in non-aqueous compositions) which contribute to the isomerization of the angelic acid ester to the tiglate ester and the acyl migration of the angelic acid moiety. To ensure an adequate chemical stability of the substance throughout the shelf-life of the composition, it should include an acidic compound capable of neutralizing alkaline impurities which may be present in one or more of the excipients of the composition and which are detrimental to the chemical stability of the ingenol derivative.

The present composition has been found to result in improved chemical stability of the ingenol derivative included therein permitting the composition to be stored at room temperature (about 25° C.) throughout its shelf-life. The improved stability may be the result of partitioning of the ingenol derivative to the lipid/oily phase of the water-in-oil emulsion due to its extremely low water solubility, thus protecting it from chemical interaction with reactive components in the aqueous phase.

Human skin, in particular the outer layer, the stratum corneum, provides an effective barrier against penetration of microbial pathogens and toxic chemicals. While this property of skin is generally beneficial, it complicates the dermal administration of pharmaceuticals in that a large quantity, if not most, of the active ingredient applied on the skin of a patient suffering from a dermal disease may not penetrate into the viable layers of the skin (the dermis and epidermis) where it exerts its activity. To ensure an adequate penetration of the active ingredient to the dermis and epidermis, it is generally preferred to include the active ingredient in a dissolved state, typically in the presence of a solvent in the form of an alcohol, e.g. ethanol or isopropanol, or a diol, e.g. propylene glycol. When used on their own as solvents, alcohols such as isopropanol and diols may give rise to significant skin irritation as they tend to dry out the skin. The drying out effect may, however, be mitigated by including an oily phase in the composition as the oil or oils may act as emollients and/or humectants. The composition may also be more easily spreadable when an oily phase is included as it evaporates less quickly than alcohols.

The present composition has been found to exhibit improved penetration of the ingenol derivative into the viable layers of the skin, but not higher permeation through the skin than seen with the hydrogel formulation disclosed in WO 2007/068963) despite containing a lower amount of an alcohol such as isopropanol as a solvent or no alcohol at all.

Furthermore, it is well known that a high concentration of surfactant in a topical composition for dermatological use may result in increased skin irritation. By providing a composition containing a low amount of surfactant and by including lipid components that are compatible with the structure of the skin, the risk of skin irritation resulting from the use of irritative excipients may be reduced.

In another aspect of the invention, the present composition may be used in the treatment of a dermal disease or condition.

### DETAILED DESCRIPTION OF THE INVENTION

### Definitions

In the present context, the term “water-in-oil emulsion” is intended to include a formulation containing an oily phase and an aqueous phase, wherein the aqueous phase is dispersed in the oily continuous phase. The ingenol derivative is present in the oily phase and in the interphase with the aqueous phase.

The term “non-ionic surfactant” is intended to indicate a surfactant comprising a hydrophilic and a hydrophobic portion in which the hydrophilic portion carries no charge but derives its surface activity from highly polar groups such as polyoxyethylene groups. For the present purpose, the surfactant may be an oil-in-water surfactant with an HLB value of 9-18 or, for compositions which contain an aqueous phase in an amount of less than about 40% by weight of the composition, the surfactant may have an HLB value of 2-12. Mixtures of surfactants with an HLB value of 9-18 and 2-12 are also contemplated.

The term “ingenol derivative” is intended to mean an ingenol compound isolated from a species of Euphorbia, in particular from E. peplus, or an ingenol derivative prepared by chemical synthesis or by a semi-synthetic route, e.g. as disclosed in copending application No. PCT/DK2011/000081. Examples of ingenol derivatives that may be included in the present compositions are ingenol-3-angelate, ingenol-5-angelate, ingenol-20-angelate, 20-O-acetyl-ingenol-3-angelate and 20-deoxy-ingenol-3-angelate. Ingenol-3-angelate, also known as ingenol-3-mebutate or PEP 005, is currently in development for the treatment of actinic keratosis.

The term “storage stability” is intended to indicate that the composition exhibits chemical and physical stability characteristics that permit storage of the composition, at refrigeration or, preferably, room temperature for a sufficient period of time (the shelf-life of the composition) to make the composition commercially viable, such as at least 12 months, in particular at least 18 months, and preferably at least 2 years.

The term “chemical stability” or “chemically stable” is intended to indicate that no more than 10%, preferably no more than 6%, of the ingenol derivative degrades over the shelf-life of the product, typically 2 years. An approximation of chemical stability at room temperature is obtained by subjecting the composition to accelerated stability studies at 40° C. If less than about 3% of the substance, e.g. ingenol-3-angelate, has degraded after 3 months at 40° C., a shelf-life of 2 years at room temperature is considered to be feasible.

The term “physical stability” or “physically stable” is intended to mean that the composition retains its macroscopic and microscopic appearance over the shelf-life of the product, e.g. that the ingenol derivative does not precipitate from the solvent phase or that there is no visible phase separation of the solvent phase and the carrier phase.

The term “solubilization capacity” is intended to indicate the ability of a solvent or mixture of solvents to dissolve a given substance, expressed as the amount required to effect complete solubilization of the substance.

The term “skin penetration” is intended to mean the diffusion of the active ingredient into the different layers of the skin, i.e. the stratum corneum, epidermis and dermis.

The term “skin permeation” is intended to mean the flux of the active ingredient through the skin into the systemic circulation or, in case of in vitro studies, the receptor fluid of the Franz cell apparatus used in the experiment.

The term “medium chain triglycerides” is intended to indicate triglyceride esters of fatty acids with a chain length of 6-12 carbon atoms. A currently favoured example of medium chain triglycerides is a mixture of caprylic (C₈) and capric (C₁₀) triglycerides, e.g. available under the trade name Miglyol 812.

The term “acidic compound” is intended to indicate a compound capable of providing a net overall acidic environment in the composition and/or capable of neutralizing alkaline impurities detrimental to the stability of the ingenol derivative.

The term “occlusive agent” is intended to indicate a lipid substance that forms a layer on the surface of the skin on application of the composition. The lipid layer forms a hydration barrier sufficient to result in reduction of transepidermal water loss, resulting in skin hydration.

### Embodiments

In the present composition, the surfactant is preferably present in a concentration of from about 1% by weight to about 8% by weight, or from about 1.5% by weight to about 7% by weight, such as about 5% by weight, of the composition.

According to the invention, the non-ionic surfactant is preferably selected from the group consisting of polyethylene glycol 8 caprylic/capric glyceride (a polyethylene glycol derivative of a mixture of mono-, di- and triglycerides of caprylic and capric acids with an average of 8 moles of ethylene oxide) or polyethylene glycol 6 caprylic/capric glyceride (a polyethylene glycol derivative of a mixture of mono-, di- and triglycerides of caprylic and capric acids with an average of 6 moles of ethylene oxide). The non-ionic surfactant is favourably polyethylene glycol 8 caprylic/capric glyceride, e.g. available from Gattefossé under the trade name Labrasol or from Condea under the trade name Softigen 767.

The non-ionic surfactant may also preferably be a polyethylene glycol C₆₋₂₀ fatty acid glyceride selected from the group consisting of caprylocaproyl PEG glyceride, lauroyl PEG glyceride, linoeoyl PEG glyceride, oleoyl PEG glyceride and stearoyl PEG glyceride, a polyoxyethylene C₈₋₂₀ alkyl ether selected from the group consisting of PEG monocetyl ether, PEG monolauryl ether, PEG monooleyl ether and PEG monostearyl ether (such as polyoxyethylene-2-stearyl ether), a polysorbate selected from the group consisting of polysorbate 20, 40, 60 and 80, or a polyoxyethylene castor oil derivative such as polyoxyl castor oil or hydrogenated polyoxyl castor oil, or a mixture of acrylamide acryloyldimethyl taurate copolymer, isohexadecane and polysorbate 80, e.g. available under the trade name SEPINEO P600, a sterol, a fatty alcohol, a fatty acid phosphate ester such as dicetyl phosphate, a mono- or diglycol ester, a mono-, di- or polyglyceryl ester such as glyceryl myristate, polyglyceryl-3-polyricinoleate, PEG-30 dipolyhydroxystearate or polyglyceryl-3-diisostearate, a mono-, di- or polyglucose ester, a sucrose ester such as sucrose cocoate, sucrose monolaurate, sucrose stearate or sucrose distearate, or a sorbitan ester such as sorbitan laurate, sorbitan palmitate, sorbitan stearate, sorbitan oleate, sorbitan sesquioleate, sorbitan trioleate or sorbitan isostearate

The composition further comprises a solvent for the ingenol derivative. In one, currently preferred embodiment, the solvent may be an oily solvent selected from a vegetable oil, e.g. sesame oil, sunflower oil, palm kernel oil, corn oil, safflower oil, olive oil, avocado oil, jojoba oil, grape kernel oil, almond oil, canola oil, coconut oil, cottonseed oil, oil, walnut oil, soybean oil or wheat germ oil, a highly purified vegetable oil, e.g. medium chain triglycerides, long chain triglycerides, castor oil, caprylic/capric mono- and diglycerides or caprylic/capric mono-, di- and triglycerides, a synthetic oil, e.g. isopropyl myristate, isopropyl palmitate, isopropyl linoleate, isopropyl monooleate, isostearyl isostearate or polyoxypropylene stearyl ether (such as polyoxypropylene-15-stearyl ether), a propylene glycol derivative such as propylene glycol dicaprylate dicaprate, and alkyl or dialkyl ester such as ethyl oleate, diisopropyle adipate or dicaprylyl carbonate, or a C₁₀₋₃₀ cholesterol or lanosterol ester.

The solvent may be present in a concentration of about 1-40%, in particular about 10-30%, or about 10-25%, or about 10-20%, or about 10-15%, by weight of the composition.

In a currently favoured embodiment, the non-ionic surfactant is polyoxyethylene-2-stearyl ether and the solvent is medium chain triglycerides.

In another embodiment, the solvent may be selected from the group consisting of lower alcohols, such as n-propanol, isopropanol, n-butanol, 2-butanol or benzyl alcohol, diols such as propylene glycol, or a mixture of one or more of these solvents which may be used on their own or as co-solvents together with an oily solvent such as one of those indicated above. Solvents of this type may also act as penetration enhancers aiding the penetration of the ingenol derivative into the viable layers of the skin. Other penetration enhancers which may be included in the present composition are glycerol, propylene carbonate, a pyrrolidone such as N-methylpyrrolidone or N-hydroxyalkylpyrrolidone, an azone, menthol, eucalyptol or nicotinamide.

In another currently favoured embodiment, the non-ionic surfactant is polyoxyethylene-2-stearyl ether and the solvent is isopropanol or a mixture of isopropanol and propylene glycol.

It is generally preferred to include the lower alcohol or diol solvent is low amounts in order to avoid or reduce skin irritation. Thus the lower alcohol or diol solvent may be present in an amount of 0.1-20% by weight, preferably 0.5-10% by weight of the composition.

The composition may further include an occlusive agent which may be selected from a mineral oil, e.g. liquid paraffin, or a hydrocarbon or mixture of hydrocarbons with chain lengths ranging from C₅ to C₆₀. A frequently used occlusive agent is petrolatum, or white soft paraffin, which is composed of hydrocarbons of different chain lengths peaking at about C₄₀₋₄₄, or a mixture of petrolatum and liquid paraffin (consisting of hydrocarbons of different chain lengths peaking at C₂₈₋₄₀). While petrolatum provides occlusion of the treated skin surface, reducing transdermal loss of water and potentiating the therapeutic effect of the active ingredient in the composition, it tends to have a greasy and/or tacky feel which persists for quite some time after application, and it is not easily spreadable. It may therefore be preferred to employ paraffins consisting of hydrocarbons of a somewhat lower chain length, such as paraffins consisting of hydrocarbons with chain lengths peaking at C₁₄₋₁₆, C₁₈₋₂₂, C₂₀₋₂₂, C₂₀₋₂₆ or mixtures thereof (the hydrocarbon composition of the paraffins has been determined by gas chromatography). It has been found that such paraffins are more cosmetically acceptable in that they are less tacky and/or greasy on application and more easily spreadable. They are therefore expected to result in improved patient compliance. Suitable paraffins of this type, termed petrolatum jelly, are manufactured by Sonneborn and marketed under the trade name Sonnecone, e.g. Sonnecone CM, Sonnecone DM1, Sonnecone DM2 and Sonnecone HV. These paraffins are further disclosed and characterized in WO 2008/141078 which is incorporated herein by reference. The occlusive agent may also be an iso-paraffin such as isohexadecane or squalane, or a silicone oil, e.g. cyclomethicone or dimethicone.

The amount of occlusive agent included in the composition may be from about 40% to about 80% by weight.

To impart a desired viscosity to the present composition, it may suitably include a lipophilic viscosity-increasing ingredient such as a wax. The wax may be a mineral wax composed of a mixture of high molecular weight hydrocarbons, e.g. saturated C₃₅₋₇₀ alkanes, such as microcrystalline wax. Alternatively, the wax may be a vegetable or animal wax, e.g. esters of C₁₄₋₃₂ fatty acids and C₁₄₋₃₂ fatty alcohols, such as beeswax or hydrogenated castor oil. Alternatively, the viscosity-increasing ingredient may be an inorganic substance such as fumed silica, e.g. available under the trade name Aerosil. The amount of viscosity-increasing ingredient may vary according to the viscosifying power of the ingredient, but may typically be in the range of about 1-20% by weight of the composition. When the viscosity-increasing ingredient is microcrystalline wax it is typically present in an amount in the range of about 5-30% by weight, e.g. about 15-20% by weight, of the composition. If the surfactant included in the composition is SEPINEO P600, it may in itself impart a suitable viscosity. SEPINEO P600 may be included in an amount of about 1-10% by weight, such as about 2.5% by weight, of the composition.

The acidic compound included in the present composition may favourably be selected from a buffer such as a citrate or acetate buffer which may be included in an amount of about 0.02-4.0% by weight of the composition, or another water-soluble acidic compound such as a hydroxy acid, e.g. lactic acod or glycolic acid. Neutralization of alkaline reacting substances may also be provided by, e.g., fumed silica, which may be included in the composition in an amount of about 3-13% by weight such as about 5-9% by weight. Alternatively, neutralization of alkaline reacting substances may be provided by addition of a fatty acid such as oleic acid, linoleic acid, stearic acid, lauric acid, palmitic acid, capric acid, caprylic acid, pelargonic acid or enanthic acid to the composition.

To maintain good physical stability of the composition, in particular to avoid separation of the aqueous and lipid phases therein, it may be advantageous to include a water-in-oil emulsifier with an HLB value of 2-8. Examples of such emulsifiers are polyoxyethylene C₈₋₂₂ alkyl ethers, e.g. polyoxyethylene stearyl ether, polyoxyethylene cetyl ether or polyoxyethylene lauryl ether.

The amount of water in the composition may range from about 1% to about 50% by weight, e.g. from about 2% to about 30% by weight or from about 2% to about 10% by weight, of the composition.

Examples of ingenol derivatives that may be included in the present composition are ingenol-3-angelate, ingenol-5-angelate, ingenol-20-angelate, 20-O-acetyl-ingenol-3-angelate and 20-deoxy-ingenol-3-angelate. A currently favoured ingenol derivative is ingenol-3-angelate, also known as ingenol-3-mebutate or PEP 005. The ingenol derivative may be included in the composition in an amount of about 0.001-0.5% by weight of the composition.

In some embodiments, the compositions of the invention are visually and behaviourally gel-like; however, these compositions are two-phase emulsions and so cannot be classified as gels.

The composition of the invention may be used in the topical treatment of a dermal disease or condition. Examples of dermal diseases and conditions are actinic keratosis, seborrheic keratosis, skin cancer, such as basal cell carcinoma or squamous cell carcinoma, warts, keloids, scars, photoaged or photodamaged skin, or acne.

The term “skin cancer” is intended to include non-melanoma skin cancer, malignant melanoma, Merkel cell carcinoma, squamous cell carcinoma or basal cell carcinoma. Basal cell carcinomas include superficial basal cell carcinoma as well as nodular basal cell carcinoma.

The term “photodamaged skin” is intended to include cover fine lines, wrinkles and UV-ageing. UV ageing is often manifested by an increase in the epidermal thickness or epidermal atrophy and most notably by solar elastosis, the accumulation of elastin containing material just below the dermal-epidermal junction. Collagen and elastic fibres become fragmented and disorganised. At a cosmetic level this can be observed as a reddening and/or thickening of the skin resulting a a leathery appearance, skin fragility and irregular pigmentation, loss of tone and elasticity, as well as wrinkling, dryness, sunspots and deep furrow formation.

The term “warts” in the context of the present invention is intended to human papilloma virus (HPV) infections leading to formation of warts on the body, such as the skin, genitals and mouth.

The present composition may also be effective at reducing or minimizing scar tissue or improving cosmesis or functional outcome in a wound and scar reduction, wherein the wound is cutaneous, chronic or for example diabetes associated, and includes cuts and lacerations, surgical incisions, punctures, graces, scratches, compression wounds, abrasions, friction wounds, chronic wounds, ulcers, thermal effect wounds, chemical wounds, wounds resulting from pathogenic infections, skin graft/transplant donor and recipient sites, immune response conditions, oral wounds, stomach or intestinal wounds, damaged cartilage or bone, amputation sides and corneal lesions.

The potency of a composition of the invention may be tested in a model where test compositions (20 μL) are applied topically, once daily, on a 2 cm² area on each flank of anaesthetized CRL:CD(SD)-HR-CD male rats (12 weeks old). 6 different animals are treated with each formulation. Animals are allowed to recover from anaesthesia after 2 hours. Dosing with formulations may vary from a single application to several, once daily applications. A visual scoring on erythema, oedema, ulceration and telangiectasia is performed 24 hrs after each application. 24 hours after the last application, animals are euthanized by asphyxiation in CO₂ and two 5 mm punch biopsies are taken from each treatment site: one biopsy is snap frozen in liquid nitrogen and stored at −20° C. until analysis for the chemokine KC (CXCL1), the other sample is fixated in formalin at room temperature for histological analysis. Clinical scoring, KC production and histological evaluation of the epidermal and dermal compartment are compared against Picato gel formulation in order to identify new formulations with the same ability to create a strong, local skin reaction, increase KC production and induce necrosis of epidermis and dermis. An increase in any of these parameters is interpreted as an increased potency of the formulation.

In a specific embodiment the composition comprises

0.0001-0.5% by weight ingenol-3-angelate
10-30% by weight medium chain triglycerides
1-10% by weight polyoxyethylene-2-stearyl ether
0.5-1.5% by weight benzyl alcohol
5-9% by weight fumed silica
2-10% by weight water buffered to pH 2.6-3.7 with citrate buffer
60-80% by weight liquid paraffin

In a further specific embodiment the composition comprises

0.0001-0.5% by weight ingenol-3-angelate
5-10% by weight isopropanol
5-10% by weight polyoxyethylene-2-stearyl ether
0.5-1.5% by weight benzyl alcohol
2-10% by weight fumed silica
2-10% by weight water buffered to pH 2.6-3.7 with citrate buffer
60-80% by weight liquid paraffin

The invention is described in further detail in the following examples which are not in any way intended to limit the scope of the invention as claimed.

### EXAMPLES

### Example 1

### Composition A

Ingenol-3-angelate 0.5 mg/g
Benzyl alcohol 9 mg/g
Citric acid 1.4 mg/g
Citrate 0.35 mg/g
Water 26 mg/g
Glycerol 100 mg/g
Polyoxyethylene-2-stearyl ether 50 mg/g
Paraffin liquid 762.75 mg/g
Aerosil 200P (amorphous anhydrous colloidal silicon dioxide) 50 mg/g

### Composition B

Ingenol-3-angelate 0.5 mg/g
Benzyl alcohol 9 mg/g
Citric acid 1.4 mg/g
Citrate 0.35 mg/g
Water 26 mg/g
Glycerol 100 mg/g
Isopropanol 100 mg/g
Polyoxyethylene-2-stearyl ether 50 mg/g
Paraffin liquid 662.75 mg/g
Aerosil 200P (amorphous anhydrous colloidal silicon dioxide) 50 mg/g

### Composition C

Ingenol-3-angelate 0.5 mg/g
Benzyl alcohol 9 mg/g
Citric acid 1.4 mg/g
Citrate 0.35 mg/g
Water 26 mg/g
Propylene glycol 100 mg/g
Isopropanol 100 mg/g
Polyoxyethylene-2-stearyl ether 50 mg/g
Paraffin liquid 662.75 mg/g
Aerosil 200P (amorphous anhydrous colloidal silicon dioxide) 50 mg/g

Compositions A-C were prepared by initially melting the surfactant (polyoxyethylene-2-stearyl ether in the oily vehicle. After cooling to room temperature, the aqueous buffer phase and the ingenol-3-angelate dissolved in benzyl alcohol were emulsified in the oily phase by homogenization. Finally, Aerosil 200P was added by moderate mixing.

### Composition D

Ingenol-3-angelate 0.5 mg/g
Benzyl alcohol 10 mg/g
Cithrol DPHS (PEG 30 Dipolyhydroxystearate) 10 mg/g
Isohexadecane 50 mg/g
Isopropyl myristate 40 mg/g
Arlamol E (PPG-15 Stearyl Ether) 30 mg/g
Glycerol 30 mg/g
Citrate buffer pH 3 829.5 mg/g

### Composition E

Ingenol-3-angelate 0.5 mg/g
Benzyl alcohol 10 mg/g
Crodafos CES (Cetearyl Alcohol, Dicetyl Phosphate and Ceteth-10 Phosphate) 50 mg/g
Isohexadecane 50 mg/g
Isopropyl myristate 40 mg/g
Arlamol E (PPG-15 Stearyl Ether) 30 mg/g
Glycerol 30 mg/g
Citrate buffer pH 3 789.5 mg/g

### Composition F

Ingenol-3-angelate 0.5 mg/g
Benzyl alcohol 10 mg/g
Emulsifying wax 50 mg/g
Isohexadecane 50 mg/g
Isopropyl myristate 40 mg/g
Arlamol E (PPG-15 Stearyl Ether) 30 mg/g
Glycerol 30 mg/g
Citrate buffer pH 3 789.5 mg/g

### Composition G

Ingenol-3-angelate 0.5 mg/g
Cithrol DPHS (PEG 30 Dipolyhydroxystearate) 20 mg/g
Isohexadecane 60 mg/g
Isopropyl myristate 50 mg/g
Arlamol E (PPG-15 Stearyl Ether) 30 mg/g
Benzyl alcohol 10 mg/g
Citrate buffer 776.5 mg/g
Glycerol 45 mg/g
MgSO₄.(H₂O)₇ 8 mg/g

### Composition H

Ingenol-3-angelate 0.5 mg/g
Cithrol DPHS (PEG 30 Dipolyhydroxystearate) 20 mg/g
Isohexadecane 60 mg/g
Isostearyl isostearate 100 mg/g
Benzyl alcohol 10 mg/g
Citrate buffer 764.5 mg/g
Glycerol 40 mg/g
MgSO₄.(H₂O)₇ 5 mg/g

### Composition I

Ingenol-3-angelate 0.5 mg/g
Arlacel 1690 (Sorbitan Isostearate and Polyglyceryl-3 Polyricinoleate) 40 mg/g
Dimethicone 50 mg/g
Isopropyl myristate 60 mg/g
Isopropylpalmitate 110 mg/g
Benzyl alcohol 10 mg/g
Citrate buffer 667 mg/g
Glycerol 40 mg/g
Cetostearyl alcohol 7.5 mg/g
Magnesium stearate 5 mg/g
MgSO₄.(H₂O)₇ 10 mg/g

### Composition J

Ingenol-3-angelate 0.5 mg/g
Arlacel 1690 (Sorbitan Isostearate and Polyglyceryl-3 Polyricinoleate) 40 mg/g
Isohexadecane 30 mg/g
Medium-chain triglycerides 50 mg/g
Isopropyl myristate 40 mg/g
Benzyl alcohol 10 mg/g
Citrate buffer 791.5 mg/g
Glycerol 30 mg/g
MgSO₄.(H₂O)₇ 8 mg/g

### Composition K

Ingenol-3-angelate 0.5 mg/g
Cithrol DPHS (PEG 30 Dipolyhydroxystearate) 10 mg/g
Propylene glycol dicaprylate dicaprate 100 mg/g
Diisopropyladipate 45 mg/g
Benzyl alcohol 10 mg/g
Citrate buffer 747.5 mg/g
Glycerol 70 mg/g
Medium-chain triglycerides 10 mg/g
MgSO₄.(H₂O)₇ 7 mg/g

### Composition L

Ingenol-3-angelate 0.5 mg/g
Crodafos CES (Cetearyl Alcohol, Dicetyl Phosphate and Ceteth-10 Phosphate) 50 mg/g
Isopropyl myristate 20 mg/g
Isostearyl isostearate 40 mg/g
Diisopropyladipate 20 mg/g
Arlamol E (PPG-15 Stearyl Ether) 20 mg/g
Benzyl alcohol 10 mg/g
Citrate buffer 764.5 mg/g
Glycerol 10 mg/g
Cetostearyl alcohol 20 mg/g
Xanthan Gum 5 mg/g
Titanium dioxide 40 mg/g

### Composition M

Ingenol-3-angelate 0.5 mg/g
Arlacel 1690 (Sorbitan Isostearate and Polyglyceryl-3 Polyricinoleate) 35 mg/g
Isohexadecane 60 mg/g
Liquid paraffin 80 mg/g
Aerosil 200P (amorphous anhydrous colloidal silicon dioxide) 20 mg/g
Medium-chain triglycerides 20 mg/g
Diisopropyladipate 20 mg/g
Benzyl alcohol 10 mg/g
Lactic acid 40 mg/g
Water 669.5 mg/g
Glycerol 40 mg/g
MgSO₄.(H₂O)₇ 5 mg/g

### Composition N

Ingenol-3-angelate 0.5 mg/g
Cithrol DPHS (PEG 30 Dipolyhydroxystearate) 10 mg/g
Isohexadecane 50 mg/g
Supersterol ester 20 mg/g
Isopropyl myristate 40 mg/g
Arlamol E (PPG-15 Stearyl Ether) 30 mg/g
Benzyl alcohol 10 mg/g
Lactic acid 40 mg/g
Water 741.5 mg/g
Glycerol 45 mg/g
MgSO₄.(H₂O)₇ 8 mg/g
Tocopheryl acetate 5 mg/g

### Composition O

Ingenol-3-angelate 0.5 mg/g
Sorbitan isostearate 25 mg/g
Aluminium stearate 1 mg/g
Light paraffin liquid 165 mg/g
Microcrystalline wax 90 mg/g
Oleyl alcohol 25 mg/g
Benzyl alcohol 10 mg/g
Citrate buffer 661.5 mg/g
Glycerol 15 mg/g
MgSO₄.(H₂O)₇ 7 mg/g

### Composition P

Ingenol-3-angelate 0.5 mg/g
Labrafil M1944 20 mg/g
Plurol diisostearique 50 mg/g
Cyclomethicone 30 mg/g
Liquid paraffin 130 mg/g
Benzyl alcohol 10 mg/g
Citrate buffer 739.5 mg/g
MgSO₄.(H₂O)₇ 10 mg/g
NaCl 10 mg/g

### Composition Q

Ingenol-3-angelate 0.5 mg/g
Plurol diisostearique 50 mg/g
Glyceryl behanate 20 mg/g
Liquid paraffin 200 mg/g
Benzyl alcohol 10 mg/g
Citrate buffer 709.5 mg/g
MgSO₄.(H₂O)₇ 5 mg/g
NaCl 5 mg/g

### Composition R

Ingenol-3-angelate 0.5 mg/g
Plurol diisostearique 40 mg/g
Plurol oleique 20 mg/g
Liquid paraffin 150 mg/g
Benzyl alcohol 10 mg/g
Citrate buffer 759.5 mg/g
MgSO₄.(H₂O)₇ 10 mg/g
NaCl 10 mg/g

### Composition S

Ingenol-3-angelate 0.5 mg/g
Plurol diisostearique 30 mg/g
Plurol oleique 20 mg/g
Dicaprylyl carbonate 220 mg/g
Squalane 30 mg/g
Benzyl alcohol 10 mg/g
Citrate buffer 659.5 mg/g
MgSO₄.(H₂O)₇ 15 mg/g
NaCl 15 mg/g

### Composition T

Ingenol-3-angelate 0.5 mg/g
Magnesium stearate 125 mg/g
Liquid paraffin 774.5 mg/g
Citrate buffer 90 mg/g
Benzyl alcohol 10 mg/g

### Composition U

Ingenol-3-angelate 0.5 mg/g
Plurol diisostearique 30 mg/g
Isopropyl-palmitate 24 mg/g
Petrolatum-Polyethylene blend Crodabase SQ 246 mg/g
Glycerol 50 mg/g
MgSO₄.(H₂O)₇ 5 mg/g
Potassium sorbate 1.4 mg/g
Citrate buffer 633.1 mg/g
Benzyl alcohol 10 mg/g

### Composition V

Ingenol-3-angelate 0.5 mg/g
Sorbitane oleate 12 mg/g
PEG 30 dipolyhydroxystearate 3 mg/g
Medium-chain triglycerides 100 mg/g
Citrate buffer 793.5 mg/g
Glycerol 61 mg/g
Sepineo P600 20 mg/g
Benzyl alcohol 10 mg/g

### Composition W

Ingenol-3-angelate 0.5 mg/g
Phospholipid Lipoid S100 400 mg/g
Liquid paraffin 399.5 mg/g
Citrate buffer 190 mg/g
Benzyl alcohol 10 mg/g

### Composition X

Ingenol-3-angelate 0.5 mg/g
Cetomacrogol emulsifying ointment 889.5 mg/g
Citrate buffer 100 mg/g
Benzyl alcohol 10 mg/g

### Composition Y

Ingenol-3-angelate 0.5 mg/g
Cetomacrogol emulsifying ointment 589.5 mg/g
Citrate buffer 400 mg/g
Benzyl alcohol 10 mg/g

### Composition Z

Ingenol-3-angelate 0.5 mg/g
Benzyl Alcohol 9 mg/g
Citric acid 1.4 mg/g
Sodium citrate 0.35 mg/g
Water 26 mg/g
Glycerol 10 mg/g
Macrogol stearyl ether 50 mg/g
Paraffin, liquid 852.75 mg/g
Aerosil 200P (amorphous anhydrous colloidal silicon dioxide) 50 mg/g

### Composition AA

Ingenol-3-angelate 0.5 mg/g
Benzyl Alcohol 9 mg/g
Citric Acid 1.4 mg/g
Sodium citrate 0.35 mg/g
Water 26 mg/g
Glycerol 100 mg/g
Macrogol stearyl 50 mg/g
Paraffin, liquid 762.75 mg/g
Aerosil 200P (amorphous anhydrous colloidal silicon dioxide) 50 mg/g

### Composition AB

Ingenol-3-angelate 0.5 mg/g
Benzyl Alcohol 9 mg/g
Citric Acid 1.4 mg/g
Sodium citrate 0.35 mg/g
Water 26 mg/g
Glycerol 100 mg/g
Isopropyl alcohol 100 mg/g
Macrogol stearyl 50 mg/g
Paraffin, liquid 662.75 mg/g
Aerosil 200P (amorphous anhydrous colloidal silicon dioxide) 50 mg/g

### Composition AC

Ingenol-3-angelate 0.5 mg/g
Benzyl Alcohol 9 mg/g
Citric Acid 1.4 mg/g
Sodium citrate 0.35 mg/g
Water 26 mg/g
Propylene glycol 100 mg/g
Macrogol stearyl 50 mg/g
Paraffin, liquid 762.75 mg/g
Aerosil 200P (amorphous anhydrous colloidal silicon dioxide) 50 mg/g

### Composition AD

Ingenol-3-angelate 0.5 mg/g
Benzyl Alcohol 9 mg/g
Citric Acid 1.4 mg/g
Sodium citrate 0.35 mg/g
Water 26 mg/g
Propylene glycol 100 mg/g
Macrogol stearyl 50 mg/g
Glycerol 100 mg/g
Paraffin, liquid 662.75 mg/g
Aerosil 200P (amorphous anhydrous colloidal silicon dioxide) 50 mg/g

### Composition AE

Ingenol-3-angelate 0.5 mg/g
Benzyl Alcohol 9 mg/g
Citric Acid 1.4 mg/g
Sodium citrate 0.35 mg/g
Water 26 mg/g
Propylene glycol 100 mg/g
Isopropyl alcohol 100 mg/g
Macrogol stearyl 50 mg/g
Paraffin, liquid 662.75 mg/g
Aerosil 200P (amorphous anhydrous colloidal silicon dioxide) 50 mg/g

### Composition AF

Ingenol-3-angelate 0.5 mg/g
Cithrol DPHS (PEG 30 Dipolyhydroxystearate) 50 mg/g
Benzyl Alcohol 10 mg/g
Citrate buffer 100 mg/g
Glycerol 100 mg/g
Liquid paraffin 689.5 mg/g
Aerosil 200P (amorphous anhydrous colloidal silicon dioxide) 50 mg/g

### Composition AG

Ingenol-3-angelate 0.5 mg/g
Plurol diisostearique 50 mg/g
Benzyl Alcohol 10 mg/g
Citrate buffer 100 mg/g
Glycerol 100 mg/g
Liquid paraffin 689.5 mg/g
Aerosil 200P (amorphous anhydrous colloidal silicon dioxide) 50 mg/g

### Composition AH

Ingenol-3-angelate 0.5 mg/g
Plurol diisostearique 50 mg/g
Benzyl Alcohol 10 mg/g
Citrate buffer 300 mg/g
Glycerol 100 mg/g
Liquid paraffin 489.5 mg/g
Aerosil 200P (amorphous anhydrous colloidal silicon dioxide) 50 mg/g

### Composition AI

Ingenol-3-angelate 0.5 mg/g
Arlacel 1690 (Sorbitan Isostearate and Polyglyceryl-3 Polyricinoleate) 50 mg/g
Benzyl Alcohol 10 mg/g
Citrate buffer 100 mg/g
Glycerol 100 mg/g
Liquid paraffin 689.5 mg/g
Aerosil 200P (amorphous anhydrous colloidal silicon dioxide) 50 mg/g

### Composition AJ

Ingenol-3-angelate 0.5 mg/g
Arlacel 1690 (Sorbitan Isostearate and Polyglyceryl-3 Polyricinoleate) 50 mg/g
Benzyl Alcohol 10 mg/g
Citrate buffer 300 mg/g
Glycerol 100 mg/g
Liquid paraffin 489.5 mg/g
Aerosil 200P (amorphous anhydrous colloidal silicon dioxide) 50 mg/g

### Composition AK

Ingenol-3-angelate 0.5 mg/g
Labrafil M 2130 25 mg/g
Citrate buffer 100 mg/g
Glycerol 100 mg/g
Benzyl Alcohol 10 mg/g
Cholesterol 10 mg/g
Stearic acid 10 mg/g
Ceramide III 5 mg/g
Liquid paraffin 639.5 mg/g
Microcrystalline wax 100 mg/g

### Composition AL

Ingenol-3-angelate 0.5 mg/g
Sisterna SP 30 25 mg/g
Citrate buffer 100 mg/g
Glycerol 100 mg/g
Benzyl Alcohol 10 mg/g
Cholesterol 10 mg/g
Stearic acid 10 mg/g
Ceramide III 5 mg/g
Liquid paraffin 639.5 mg/g
Microcrystalline wax 100 mg/g

### Composition AM

Ingenol-3-angelate 0.5 mg/g
Benzyl alcohol 10 mg/g
Citrate buffer pH 3.0 50 mg/g
Dow Corning® BY 11-030 45 mg/g
Dow Corning® ST-Elastomer 100 mg/g
Dow Corning® ST cyclomethicone 5-NF 155 mg/g
Propylene glycol 589.5 mg/g
Ethanol 58 mg/g

### Composition AN

PEP005 0.5 mg/g
PEG 30 Dipolyhydroxystearate (Cithrol DPHS) 50 mg/g
Benzyl alcohol 10 mg/g
Citrate buffer 27.75 mg/g
Glycerol 100 mg/g
Paraffin liquid 761.75 mg/g
Aerosil 200P 50 mg/g

### Composition AO

PEP005 0.5 mg/g
Labrafil M 1944 50 mg/g
Benzyl alcohol 10 mg/g
Citrate buffer 27.75 mg/g
Glycerol 100 mg/g
Paraffin liquid 761.75 mg/g
Aerosil 200P 50 mg/g

### Composition AP

PEP005 0.5 mg/g
Span 120 50 mg/g
Benzyl alcohol 10 mg/g
Citrate buffer 27.75 mg/g
Glycerol 100 mg/g
Paraffin liquid 761.75 mg/g
Aerosil 200P 50 mg/g

### Composition AQ

PEP005 0.5 mg/g
Glyceryl monooleate 50 mg/g
Benzyl alcohol 10 mg/g
Citrate buffer 27.75 mg/g
Glycerol 100 mg/g
Paraffin liquid 761.75 mg/g
Aerosil 200P 50 mg/g

### Composition AR

PEP005 0.5 mg/g
Caprylic/capric glycerides (Akoline MCM) 50 mg/g
Benzyl alcohol 10 mg/g
Citrate buffer 27.75 mg/g
Glycerol 100 mg/g
Paraffin liquid 761.75 mg/g
Aerosil 200P 50 mg/g

### Composition AS

PEP005 0.5 mg/g
Plurol diisostearique 50 mg/g
Benzyl alcohol 10 mg/g
Citrate buffer 27.75 mg/g
Glycerol 100 mg/g
Paraffin liquid 761.75 mg/g
Aerosil 200P 50 mg/g

### Composition AT

PEP005 0.5 mg/g
Arlacel 1690 50 mg/g
Benzyl alcohol 10 mg/g
Citrate buffer 27.75 mg/g
Glycerol 100 mg/g
Paraffin liquid 761.75 mg/g
Aerosil 200P 50 mg/g

### Composition AU

PEP005 0.5 mg/g
Polawax NF50 mg/g
Benzyl alcohol 10 mg/g
Citrate buffer 27.75 mg/g
Glycerol 100 mg/g
Paraffin liquid 761.75 mg/g
Aerosil 200P 50 mg/g

### Composition AV

PEP005 0.5 mg/g
Crodafos CES 50 mg/g
Benzyl alcohol 10 mg/g
Citrate buffer 27.75 mg/g
Glycerol 100 mg/g
Paraffin liquid 761.75 mg/g
Aerosil 200P 50 mg/g

### Composition AW

PEP005 0.5 mg/g
Cithrol DPHS 25 mg/g
Citrate buffer 27.75 mg/g
Glycerol 100 mg/g
Benzylalcohol 10 mg/g
Cholesterol 10 mg/g
Stearic acid 10 mg/g
Ceramide III. 5 mg/g
Liquid paraffin 711.75 mg/g
Microcrystalline wax 100 mg/g

### Composition AX

PEP005 0.5 mg/g
Labrafil M 2130 25 mg/g
Citrate buffer 27.75 mg/g
Glycerol 100 mg/g
Benzylalcohol 10 mg/g
Cholesterol 10 mg/g
Stearic acid 10 mg/g
Ceramide III. 5 mg/g
Liquid paraffin 711.75 mg/g
Microcrystalline wax 100 mg/g

### Composition AY

PEP005 0.5 mg/g
Span 120 25 mg/g
Citrate buffer 27.75 mg/g
Glycerol 100 mg/g
Benzylalcohol 10 mg/g
Cholesterol 10 mg/g
Stearic acid 10 mg/g
Ceramide III. 5 mg/g
Liquid paraffin 711.75 mg/g
Microcrystalline wax 100 mg/g

### Composition AZ

PEP005 0.5 mg/g
Caprylic/capric glycerides (Akoline MCM) 25 mg/g
Citrate buffer 27.75 mg/g
Glycerol 100 mg/g
Benzylalcohol 10 mg/g
Cholesterol 10 mg/g
Stearic acid 10 mg/g
Ceramide III. 5 mg/g
Liquid paraffin 711.75 mg/g
Microcrystalline wax 100 mg/g

### Composition BA

PEP005 0.5 mg/g
Labrafil M 2130 25 mg/g
Citrate buffer 27.75 mg/g
Glycerol 100 mg/g
Benzylalcohol 10 mg/g
Cholesterol 10 mg/g
Stearic acid 10 mg/g
Ceramide III. 5 mg/g
Liquid paraffin 711.75 mg/g
Microcrystalline wax 100 mg/g

### Composition BB

PEP005 0.5 mg/g
Sisterna SP30 25 mg/g
Citrate buffer 27.75 mg/g
Glycerol 100 mg/g
Benzylalcohol 10 mg/g
Cholesterol 10 mg/g
Stearic acid 10 mg/g
Ceramide III. 5 mg/g
Liquid paraffin 711.75 mg/g
Microcrystalline wax 100 mg/g

### Composition BC

PEP005 0.5 mg/g
Cetostearyl alcohol 25 mg/g
Citrate buffer 27.75 mg/g
Glycerol 100 mg/g
Benzylalcohol 10 mg/g
Cholesterol 10 mg/g
Stearic acid 10 mg/g
Ceramide III. 5 mg/g
Liquid paraffin 711.75 mg/g
Microcrystalline wax 100 mg/g

The compositions were tested for chemical stability by extracting ingenol-3-angelate from the composition by dissolution in a solvent mixture of acetonitrile and phosphoric acid. Identification, assay and determination of organic impurities were determined by reversed phase HPLC with UV detection at 220 nm. Compositions A-F, H-Q, U, X, Z, AA, AF, AH, AI, AK, AM, AX, AY, AZ, BB and BC were found to be stable after 3 months at 40° C., indicating that the compositions are likely to have a shelf life for about 2 years at room temperature.

### Example 2

### Results of Skin Penetration Studies

To investigate the skin penetration and permeation of ingenol-3-angelate from compositions of the invention, a skin diffusion experiment was conducted. Full thickness skin from pig ears was used in the study. The ears were kept frozen at −18° C. before use. On the day prior to the experiment the ears were placed in a refrigerator (5±3° C.) for slow defrosting. On the day of the experiment, the hairs were removed using a veterinary hair trimmer. The skin was cleaned for subcutaneous fat using a scalpel and two pieces of skin were cut from each ear and mounted on Franz diffusion cells in a balanced order.

Static Franz-type diffusion cells with an available diffusion area of 3.14 cm² and receptor volumes ranging from 8.6 to 11.1 ml were used in substantially the manner described by T. J. Franz, “The finite dose technique as a valid in vitro model for the study of percutaneous absorption in man”, in Current Problems in Dermatology, 1978, J. W. H. Mall (Ed.), Karger, Basel, pp. 58-68. The specific volume was measured and registered for each cell. A magnetic bar was placed in the receptor compartment of each cell. After mounting the skin, physiological saline (35° C.) was filled into each receptor chamber for hydration of the skin. The cells were placed in a thermally controlled water bath which was placed on a magnetic stirrer set at 400 rpm. The circulating water in the water baths was kept at 35±1° C. resulting in a temperature of about 32° C. on the skin surface. After one hour the saline was replaced by receptor medium, 0.04 M isotonic phosphate buffer, pH 7.4 (35° C.), containing 4% bovine serum albumin. Sink conditions were maintained at all times during the period of the study, i.e. the concentration of the active compounds in the receptor medium was below 10% of the solubility of the compounds in the medium.

The in vitro skin permeation of Composition C of Example 1 and a hydrogel composition according to WO 2007/068963 was tested in 6 replicates (i.e. n=6). Each test composition was applied to the skin membrane at 0 hours in an intended dose of 4 mg/cm². A glass spatula was used for the application, and the residual amount of the composition was determined so as to give the amount of the composition actually applied on the skin.

The skin penetration experiment was allowed to proceed for 21 hours. Samples were then collected from the following compartments:

The stratum corneum was collected by tape stripping 10 times using D-Squame® tape (diameter 22 mm, CuDerm Corp., Dallas, Tex., USA). Each tape strip is applied to the test area using a standard pressure for 5 seconds and removed from the test area in one gentle, continuous move. For each repeated strop, the direction of tearing off was varied. The viable epidermis and dermis was then sampled from the skin in a similar fashion.

Samples (1 ml) of the receptor fluid remaining in the diffusion cell were collected and analysed.

The concentration of ingenol-3-angelate in the samples were determined by LC mass spectrometry.

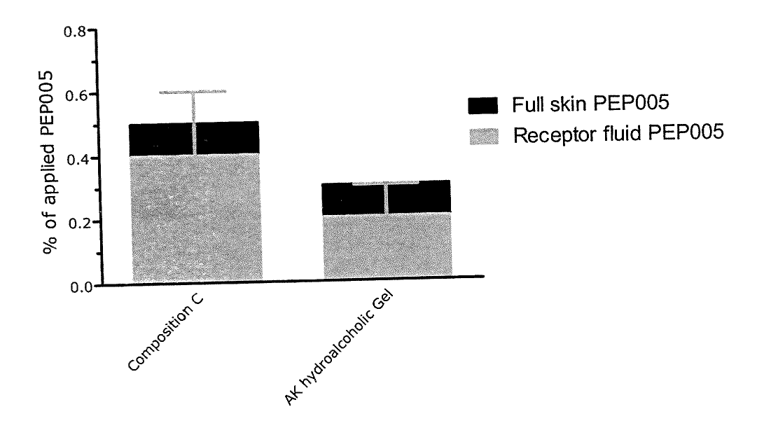

The results appear from FIG. 1 below which shows the amount of ingenol-3-angelate found in viable skin (dermis and epidermis) and receptor fluid in % of the applied dose of Composition C compared to the hydrogel formulation disclosed in WO 2007/068963. It appears from the FIGURE that the total amount of ingenol-3-angelate permeating through skin after application of Composition C is significantly higher than the amount permeating from the hydrogel formulation.

1. A topical composition for cutaneous application which is a water-in-oil emulsion comprising an oily phase comprising
(a) an ingenol derivative in dissolved form;
(b) at least one non-ionic surfactant selected from the group consisting of polyoxyl glycerides, polyoxyethylene castor oil derivatives, polyoxyethylene alkyl ethers, polysorbates, or a mixture of acrylamide acryloyldimethyl taurate copolymer, isohexadecane and polysorbate 80, sterols, fatty alcohols, fatty acid phosphonates, mono- or diglycol esters, mono- di- or polyglyceryl esters, mono-, di- or plyglucose esters, sucrose esters or sorbitan esters, the non-ionic surfactant being present in an amount of from about 0.5% by weight to about 10% by weight of the composition;
(c) a solvent for the ingenol derivative; and
an aqueous phase buffered to a pH of 2.6-3.7.

2. The composition according to claim 1, wherein the ingenol derivative is selected from the group consisting of ingenol-3-angelate, ingenol-5-angelate, ingenol-20-angelate, 20-O-acetyl-ingenol-3-angelate and 20-deoxy-ingenol-3-angelate.

3. A composition according to claim 2, wherein the ingenol derivative is ingenol-3-angelate.

4. The composition according to claim 1, wherein the non-ionic surfactant is present in a total concentration of from about 1% by weight to about 8% by weight, or from about 1.5% by weight to about 7% by weight, such as about 5% by weight, of the composition.

5. The composition according to claim 1, wherein the non-ionic surfactant is a polyethylene glycol C₆₋₂₀ fatty acid glyceride selected from the group consisting of caprylocaproyl PEG glyceride, lauroyl PEG glyceride, linoeoyl PEG glyceride, oleoyl PEG glyceride and stearoyl PEG glyceride, a polyoxyethylene C₈₋₂₀ alkyl ether selected from the group consisting of PEG monocetyl ether, PEG monolauryl ether, PEG monooleyl ether and PEG monostearyl ether, a polysorbate selected from the group consisting of polysorbate 20, 40, 60 and 80, a polyoxyethylene castor oil derivative such as polyoxyl castor oil or hydrogenated polyoxyl castor oil, or a mixture of acrylamide acryloyldimethyl taurate copolymer, isohexadecane and polysorbate 80, a sterol, a fatty alcohol, a fatty acid phosphate ester such as dicetyl phosphate, a mono- or diglycol ester, a mono-, di- or polyglyceryl ester such as glyceryl myristate, polyglyceryl-3-polyricinoleate, PEG-30 dipolyhydroxystearate or polyglyceryl-3-diisostearate, a mono-, di- or polyglucose ester, a sucrose ester such as sucrose cocoate, sucrose monolaurate, sucrose stearate or sucrose distearate, or a sorbitan ester such as sorbitan laurate, sorbitan palmitate, sorbitan stearate, sorbitan oleate, sorbitan sesquioleate, sorbitan trioleate or sorbitan isostearate.

6. The composition according to claim 1 wherein the non-ionic surfactant is polyoxyethylene-2-stearyl ether.

7. The composition according to claim 1, wherein the solvent is an oily solvent selected from the group consisting of a vegetable oil, e.g. sesame oil, sunflower oil, palm kernel oil, corn oil, safflower oil, olive oil, avocado oil, jojoba oil, grape kernel oil, almond oil, canola oil, coconut oil, cottonseed oil, peanut oil, walnut oil, soybean oil or wheat germ oil a highly purified vegetable oil, e.g. medium chain triglycerides, long chain triglycerides, castor oil, caprylic/capric mono- and diglycerides or caprylic/capric mono-, di- and triglycerides, a synthetic oil, e.g. isopropyl myristate, isopropyl palmitate, isopropyl linoleate, isopropyl monooleate, isostearyl isostearate or polyoxypropylene stearyl ether, a propylene glycol derivative such as propylene glycol dicaprylate dicaprate, and alkyl or dialkyl ester such as ethyl oleate, diisopropyle adipate or dicaprylyl carbonate, or a C₁₀₋₃₀ cholesterol or lanosterol ester.

8. The composition according to claim 7, wherein the oily solvent is present in a concentration of about 5-40%, in particular about 10-30%, or about 10-25%, or about 10-20%, or about 10-15%, by weight of the composition.

9. The composition according to claim 1 wherein the solvent is selected from the group consisting of lower alcohols, such as n-propanol, isopropanol, n-butanol, 2-butanol or benzyl alcohol, or diols such as propylene glycol.

10. The composition according to claim 1 further comprising a penetration enhancer such as glycerol, propylene carbonate, a pyrrolidone such as N-methylpyrrolidone or N-hydroxyalkylpyrrolidone, an azone, menthol, eucalyptol or nicotinamide.

11. The composition according to claim 1, wherein the solvent is medium chain triglycerides and the non-ionic surfactant is polyoxyethylene-2-stearyl ether, or wherein the solvent is isopropanol or a mixture of isopropanol and propylene glycol and the non-ionic surfactant is polyoxyethylene-2-stearyl ether or SEPINEO P600

12. A composition according to claim 1 further comprising an occlusive agent selected from the group consisting of a mineral oil, e.g. liquid paraffin, or at least one paraffin selected from paraffins consisting of hydrocarbons with chain lengths from C₅ to C₆₀, the chain lengths peaking at C₁₄₋₁₆, C₁₈₋₂₂, C₂₀₋₂₂, C₂₀₋₂₆, C₂₈₋₄₀, and C₄₀₋₄₄, or mixtures thereof or an iso-paraffin such as isohexadecane or squalane, or a silicone oil, e.g. cyclomethicone or dimethicone.

13. A composition according to claim 1, further comprising a viscosity-increasing ingredient.

14. A composition according to claim 11, wherein the viscosity-increasing ingredient is a wax, e.g. microcrystalline wax, silicone wax or hydrogenated castor oil, or fumed silica.

15. A composition according to claim 1, wherein the aqueous phase comprises 1-40% by weight, such as 2-30% by weight or 2-10% by weight, of the composition.

16. A composition according to claim 1, further comprising an acidic compound.

17. A composition according to claim 16, wherein said compound is fumed silica, or a fatty acid such as oleic acid, linoleic acid, stearic acid lauric acid, palmitic acid, capric acid, caprylic acid, pelargonic acid or enanthic acid.

18. A composition according to claim 1 comprising about 0.001-0.5% by weight of the ingenol derivative.

19. A composition according to claim 1 comprising
0.0001-0.5% by weight ingenol-3-angelate
10-30% by weight medium chain triglycerides
1-10% by weight polyoxyethylene-2-stearyl ether
0.5-1.5% by weight benzyl alcohol
5-9% by weight fumed silica
2-10% by weight water buffered to pH 2.6-3.7 with citrate buffer
40-80% by weight liquid paraffin, or
0.0001-0.5% by weight ingenol-3-angelate
5-10% by weight isopropanol
5-10% by weight polyoxyethylene-2-stearyl ether
0.5-1.5% by weight benzyl alcohol
5-9% by weight fumed silica
2-10% by weight water buffered to pH 2.6-3.7 with citrate buffer
60-80% by weight liquid paraffin

20. A composition according to claim 1 for use in the treatment of a dermal disease or condition.

21. The composition of claim 20, wherein the dermal disease or condition is actinic keratosis, seborrheic keratosis, basal cell carcinoma, squamous cell carcinoma, warts, keloids, scars, photoaged or photodamaged skin, or acne.
